(19) United States
(12) Patent Application Publication
(10) Pub. No.: US 2023/0372142 A1
(43) Pub. Date: Nov. 23, 2023

(54) SUCTION CATHETER

(71) Applicant: Tracy D. Baker, Concord, CA (US)

(72) Inventor: Tracy D. Baker, Concord, CA (US)

(21) Appl. No.: 18/199,507

(22) Filed: May 19, 2023

Related U.S. Application Data
(60) Provisional application No. 63/344,182, filed on May 20, 2022.

(51) Int. Cl. A61F 5/453; A61F 5/44

(52) U.S. Cl. CPC A61F 5/453; A61F 5/4401; A61F 5/4405; A61F 5/4408

(57) ABSTRACT

Disclosed herein are embodiments of an external catheter that may be made of soft and flexible medical-grade silicone (or other suitable) material. The catheter is designed to be gentle on the skin and easy to use for men who need a non-invasive option for managing urinary incontinence. The silicone material used may be hypoallergenic, latex-free, and not contain any phthalates or BPA. Moreover, it may be securely attached to a person without requiring any shaving of the affected area. This provides for an easy attachment and for quick removal.

### PRIORITY

This application claims the benefit of provisional patent application No. 63/344,182 filed May 20, 2022, by the same inventor and is incorporated by reference, together with its appendix, as if it were fully set forth herein.

### BACKGROUND

Historically condom catheters have existed for males that require the condom catheter to be placed around the penis. However, physiological affects can make securing the condom catheter difficult. The result is that many males acquire moisture related skin breakdown, so medical professionals opt for an insertable catheter instead. Insertable catheters are expensive and require care when inserting and removing and carry an increased risk of catheter acquired infection, which is extremely expensive to treat.

Existing techniques for urinary incontinence include difficulty in insertion, (including associated invasive procedures), time consuming application such as having to shave the affected area, more requisite skill of the medical profession, and laborious and time-consuming applications. Accordingly, there is a need to an affordable, easy-to-use, reliable suction catheter.

### BRIEF DESCRIPTION OF THE DRAWINGS

FIG. 1 shows a sketch of certain elements according to the current disclosure.

FIG. 2 shows an embodiment applied to a user.

### SUMMARY

Disclosed herein are embodiments of an external catheter that may be made of soft and flexible medical-grade silicone (or other suitable) material. The catheter is designed to be gentle on the skin and easy to use for men who need a non-invasive option for managing urinary incontinence. The silicone material used may be hypoallergenic, latex-free, and not contain any phthalates or BPA. Moreover, it may be securely attached to a person without requiring any shaving of the affected area. This provides for an easy attachment and for quick removal.

### Description

### Generality of Invention

This application should be read in the most general possible form. This includes, without limitation, the following:

References to specific techniques include alternative and more general techniques, especially when discussing aspects of the invention, or how the invention might be made or used.

References to “preferred” techniques generally mean that the inventor contemplates using those techniques, and thinks they are best for the intended application. This does not exclude other techniques for the invention, and does not mean that those techniques are necessarily essential or would be preferred in all circumstances.

References to contemplated causes and effects for some implementations do not preclude other causes or effects that might occur in other implementations.

References to reasons for using particular techniques do not preclude other reasons or techniques, even if completely contrary, where circumstances would indicate that the stated reasons or techniques are not as applicable.

Furthermore, the invention is in no way limited to the specifics of any particular embodiments and examples disclosed herein. Many other variations are possible which remain within the content, scope and spirit of the invention, and these variations would become clear to those skilled in the art after perusal of this application.

### Lexicography

The terms “effect”, “with the effect of” (and similar terms and phrases) generally indicate any consequence, whether assured, probable, or merely possible, of a stated arrangement, cause, method, or technique, without any implication that an effect or a connection between cause and effect are intentional or purposive.

The term “relatively” (and similar terms and phrases) generally indicates any relationship in which a comparison is possible, including without limitation “relatively less”, “relatively more”, and the like. In the context of the invention, where a measure or value is indicated to have a relationship “relatively”, that relationship need not be precise, need not be well-defined, need not be by comparison with any particular or specific other measure or value. For example and without limitation, in cases in which a measure or value is “relatively increased” or “relatively more”, that comparison need not be with respect to any known measure or value, but might be with respect to a measure or value held by that measurement or value at another place or time.

The term “substantially” (and similar terms and phrases) generally indicates any case or circumstance in which a determination, measure, value, or otherwise, is equal, equivalent, nearly equal, nearly equivalent, or approximately, what the measure or value is recited. The terms “substantially all” and “substantially none” (and similar terms and phrases) generally indicate any case or circumstance in which all but a relatively minor amount or number (for “substantially all”) or none but a relatively minor amount or number (for “substantially none”) have the stated property. The terms “substantial effect” (and similar terms and phrases) generally indicate any case or circumstance in which an effect might be detected or determined.

The terms “this application”, “this description” (and similar terms and phrases) generally indicate any material shown or suggested by any portions of this application, individually or collectively, and include all reasonable conclusions that might be drawn by those skilled in the art when this application is reviewed, even if those conclusions would not have been apparent at the time this application is originally filed.

### DETAILED DESCRIPTION

Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

One objective of the present disclosure is an external catheter that is made of soft and flexible medical-grade silicone (or other suitable) material. The catheter is designed to be gentle on the skin and easy to use for men who need a non-invasive option for managing urinary incontinence. The silicone material used may be hypoallergenic, latex-free, and not contain any phthalates or BPA. Moreover, it may be securely attached to a person without requiring any shaving of the affected area, and easily attached for quick application or removal.

### Cavity

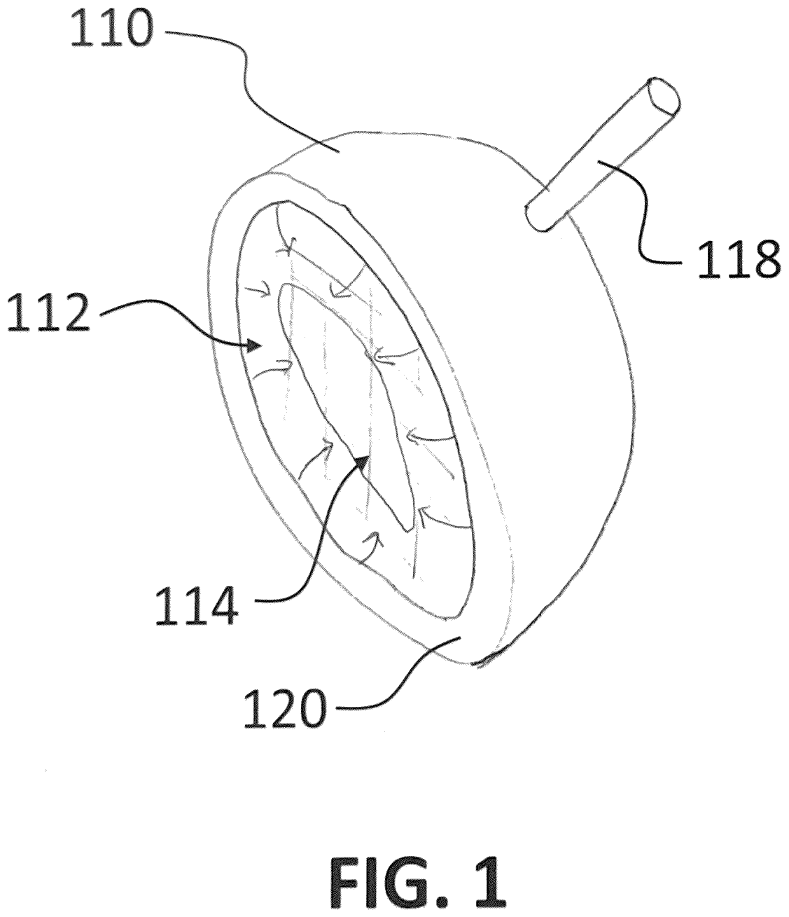

FIG. 1 illustrates certain elements of the current disclosure. In FIG. 1 a water-proof vessel 110 is formed to include an inner cavity 112. Inside the inner cavity 112 is a sponge material 114 which may occupy a majority of the cavity 112. Also attached to the water-proof vessel 110 is a hollow tube (or hose) which is disposed through the water-proof vessel 110 into the cavity 112.

The water-proof vessel 110 may be made from any bio-compatible material such as plastic silicone and the like. As shown, the water-proof structure 110 has a lip (or rim) 120 about an outer surface of the cavity 112. This rim 120 is designed to be placed on human skin to create a water-proof seal.

The tubing 118 may be formed from any suitable material for removing liquid. Conventional tubing is formed from DEHP-free PVC, TPE, FEP, high-density polyethylene, platinum-cured silicone, and peroxide-cured silicone, and the like. The tubing 118 is disposed into the cavity 112 and may be slidably inserted to allow for re-positioning the tubing 118 if desired. In some embodiments the tubing 118 is secured to the water-proof vessel 110.

### Operation

In operation, the water-proof vessel 110 with the sponge 114 inserted into the cavity 112 would be attached to a securing mechanism (not shown) such as a cloth band, modified athletic supporter and the like. A modified athletic supporter may provide a pouch for holding the vessel 110 in place. The securing mechanism would be attached to a person's body with straps, VELCRO closures, or other attachments, and disposed to allow the vessel 110 to be placed over a penis. The vessel 110 may be disposed to allow for urination without spillage through the rim 120. Suction may be applied using the tube 118 inserted through the vessel 110 and into the cavity 112 to remove urine as it is excreted. Accordingly, VELCRO or an adhesive may be disposed near the tube 118 to secure the tube to the vessel 110.

References in the specification to “one embodiment”, “an embodiment”, “an example embodiment”, etc., indicate that the embodiment described may include a particular feature, structure or characteristic, but every embodiment may not necessarily include the particular feature, structure or characteristic. Moreover, such phrases are not necessarily referring to the same embodiment. Further, when a particular feature, structure or characteristic is described in connection with an embodiment, it is submitted that it is within the knowledge of one of ordinary skill in the art to effect such feature, structure or characteristic in connection with other embodiments whether or not explicitly described. Parts of the description are presented using terminology commonly employed by those of ordinary skill in the art to convey the substance of their work to others of ordinary skill in the art.

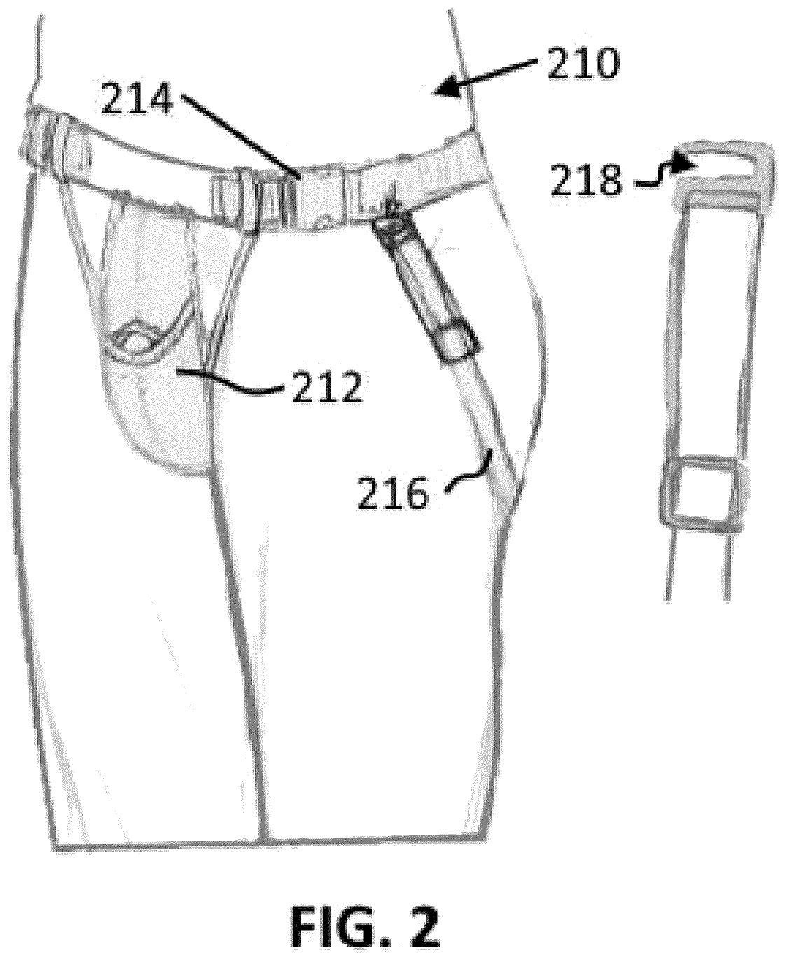

FIG. 2 shows an embodiment of the current disclosure applied to a user 210. Water-proof vessel with sponge is secured in a pouch 212. The pouch 212 is secured to a user 210 using belt 214 to secure the belt 214 around the user 210. The belt 214 may be formed from cloth, paper, plastic, or other suitable material. Additional support may be provided by one or more strap 216 to supply additional support for water-tight operation.

The belt 214 holding the pouch 212 may be secures using clips 218, adhesives, VELCO, or other suitable fastening material. The pouch 212 may have a hole for access to a tube (not shown) for connection to a vacuum pump.

In some embodiments securing the vessel 110 may be effectuated by means such as removeable straps, VELCRO straps, and the like. In operation, the vessel may be secured tight enough to effectuate a water-tight seal out of the cavity 112 when the vessel 110 is disposed on a person. Some embodiments may use paper straps with adhesive already applied. This will allow for a user to peel off a protective layer exposing the adhesive portion of the strap. Then the strap may be fitted to the user and held in place by the adhesive coating.

In another representative embodiment, straps with VELCRO may be employed for rapid installation of the vessel 110.

Certain embodiments may be effectuated using the information in the attached appendix which is incorporated by reference as if fully set forth herein.

The above illustration provides many different embodiments or embodiments for implementing different features of the invention. Specific embodiments of components and processes are described to help clarify the invention. These are, of course, merely embodiments and are not intended to limit the invention from that described in the claims.

Although the invention is illustrated and described herein as embodied in one or more specific examples, it is nevertheless not intended to be limited to the details shown, since various modifications and structural changes may be made therein without departing from the spirit of the invention and within the scope and range of equivalents of the claims. Accordingly, it is appropriate that the appended claims be construed broadly and in a manner consistent with the scope of the invention, as set forth in the following claims.

What is claimed:

1. A device including:
a vessel with a cavity;
a sponge disposed in the cavity
a securing device attached to the vessel and operable to secure the vessel to a human body, and
a suction hosed disposed in the cavity.
